(19) United States
(12) Patent Application Publication
(10) Pub. No.: US 2025/0319055 A1
(43) Pub. Date: Oct. 16, 2025

(54) COMPOSITIONS FOR TREATING GENITAL HERPES AND METHODS OF THEIR USE

(71) Applicant: PAGARI LIFE SCIENCE CORP, Bethalto, IL (US)

(72) Inventors: Alan WICKENHAUSER, Bethalto, IL (US); Stephen PEIPERT, Edwardsville, IL (US); David FELDKER, Waterloo, IL (US)

(21) Appl. No.: 19/026,003

(22) Filed: Jan. 16, 2025

Related U.S. Application Data
(60) Provisional application No. 63/632,462, filed on Apr. 10, 2024.

(51) Int. Cl. A61K 31/245; A61K 9/00; A61K 47/10; A61P 31/22

(52) U.S. Cl. CPC A61K 31/245; A61K 9/0014; A61K 47/10; A61P 31/22

(57) ABSTRACT

This disclosure provides compositions and method of treating subjects having genital herpes arising from HSV-2 viral pathogens. In particular, the compositions of this disclosure include tetracaine formulations configured for topical application to ulcers arising from genital herpes.

### PRIOR RELATED APPLICATIONS

This application claims priority to U.S. Provisional Application Ser. No. 63/632,462, filed Apr. 10, 2024, the contents of which are herein incorporated in their entirety.

### FIELD

This disclosure relates to methods and compositions for treating genital herpes. In particular this disclosure includes compositions comprising a Ryanodine receptor antagonist (e.g., tetracaine) formulations which can treat HSV2-expressing subjects, kill or reduce the incidence of the HSV2 virus, and/or reduce the number of genital ulcers caused by genital herpes in a subject.

### TECHNICAL BACKGROUND

The following includes information that may be useful in understanding the present invention. It is not an admission that any of the information, publications or documents specifically or implicitly referenced herein is prior art, or essential, to the presently described or claimed inventions. All publications, patents, related applications, and other written or electronic materials mentioned or identified herein are hereby incorporated herein by reference in their entirety. The information incorporated is as much a part of the application as filed as if all of the text and other content was repeated in the application, and should be treated as part of the text and content of the application as filed.

Herpes simplex virus (HSV), e.g., herpes simplex virus type 1 (HSV-1) and herpes simplex virus type 2 (HSV-2), is a highly contagious viral pathogen. HSV-1 generally causes intermittent, painful blistering of the mouth and mucous membranes. HSV-2 generally causes intermittent, painful blistering in the genital region. HSV can cause lifelong, recurring bouts of viral reactivity.

Genital herpes is the main cause of genital ulcers worldwide; the prevalence of herpes simplex virus (HSV) type 2 infections in the general population ranges from 10% to 60%. Most genital herpes is caused by HSV-2, although HSV-1 accounts for about half of new cases of genital herpes in developed countries (Gupta et al., Lancet, 370 (9605), P2127-2137, (2007)). Infection with HSV-1 and/or HSV-2 is permanent. After initial infection with HSV-1 or HSV-2, the virus establishes latent infection that lasts for the lifetime of the host. Following establishment of latent infection, reactivation of HSV-2 can occur at any point during the lifetime of the subject. Primary HSV-2 infections can be treated with antiviral therapy, including acyclovir (Sitavig™ or Zovirax™), valacyclovir (Valtrex™) and famciclovir (Famvir™). These therapies may reduce viral shedding, decrease pain and improve healing time of lesions.

HSV-2, unlike, despite causing similar blistering conditions, differs significantly from VZV (Varicella Zoster Virus, the pathology of which is commonly referred to as Shingles) at the molecular level. HSV-2 and HSV-1 have much larger genomes compared to VZV. The genome of HSV-2 is over 152 kilobase pairs (kbp), while the genome of VZV is only around 129 kbp. Although both are herpes viruses, they have a distant evolutionary relationship. This is reflected in the significant difference in genome size and overall lower genetic homology compared to HSV-1 and HSV-2. The percentage of guanine (G) and cytosine (C) nucleotides differs. HSV-1 and HSV-2 have a higher G+C content (around 68%) compared to VZV (around 46%). This indicates a substantial number of mutations accumulated since their evolutionary divergence. Also, certain genes are present in HSV-1 and HSV-2 but not in VZV. For example, the UL 8.5 gene found in HSV-2 has no clear equivalent in VZV. In addition to the molecular structural differences, the two viruses also differ in performance. HSV-1 exhibits a higher recombination rate compared to that of VZV, further highlighting their distinct evolutionary paths.

Vaccines are in development for the prevention of HSV-1 and HSV-2 infections. However, the reported vaccination efficacy has been limited—with only 35% effectiveness in preventing HSV-1 infections (Belshe et al., 2012; New England Journal of Medicine 366(1): 34-43). Some HSV vaccines have unfortunately been reported to exhibit significant side effects, including Gaucher's disease.

Despite advances in antiretroviral therapies, there remains a need for the treatment, prevention and/or reduction of HSV-2 infections, particularly the treatment, prevention and/or reduction of HSV-2 associated genital ulcers which are painful and decrease a patient's quality of life.

### BRIEF SUMMARY

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this introduction, which is included for purposes of illustration only and not restriction.

It is an object of the invention to provide compounds, compositions, formulations, kits and methods for the treatment of HSV-2 (herpes simplex virus-2). Thus, in one aspect, the present invention relates to methods for treating HSV-2 associated genital herpes in a subject, the method comprising topically administering a therapeutically effective amount of a composition comprising one or a plurality of Ryanodine receptor antagonists to the subject. In one aspect, the present invention relates to methods for reducing the number of ulcers associated with genital herpes in a subject, the method comprising topically administering a therapeutically effective amount of a composition comprising one or a plurality of Ryanodine receptor antagonists to the subject. In one aspect, the present invention relates to methods of inhibiting the replication of a HSV-2 in a subject, the method comprising administering a therapeutically effective amount of a composition comprising one or a plurality of Ryanodine receptor antagonists to the subject. In some aspects, the composition can comprise a non-aqueous vehicle. In some aspects, the number of ulcers can be reduced, e.g. to zero, within five days after the initial administration of the composition. In some aspects, the composition can be topically applied up to three times a day. In some aspects, the composition can be topically applied twice daily. In some aspects, the subject presents a herpes ulcer and the composition can be topically applied to said ulcer.

In some aspects, the present invention relates to compositions which can include a Ryanodine receptor antagonist selected from: tetracaine, procaine, dantrolene, chlorantraniliprole, cyantraniliprole, flubendiamide, cyclaniliprole, tetraniliprole, ryanodine, JTV 519 fumarate ((4-[3(1-(4-benzyl)piperidinyl)propionyl]-7-methoxy-2,2,4,5-tetrahydro-1,4-benzothiazepine, fumarate salt), ruthenium red ([(NH₃)₅RuORu(NH₃)₄ORu(NH₃)₅]Cl₆), DHBP (1,1′-diheptyl-4,4′-bipyridium), VK-II-86, Phenytoin (diphenylhydantoin), Flecainide, Carvedilol, EL20 (2-(diethylamino)ethyl 4-(butylamino)-2-methoxybenzoate), Xanthotoxol, 5-hydroxy-1,4-naphthalenedione, Rycal (ARM210) (Benzoic acid, 4-((2,3-dihydro-7-methoxy-1,4-benzothiazepin-4(5H)-yl)methyl)-), 4-(2-aminopropyl)-3,5-dichloro-N,N-dimethylaniline (FLA 365), 2-(dimethylamino)ethyl 4-(butylamino)-5-chloro-2-methoxybenzoate (EL1), 2-(dibutylamino)ethyl 4-(butylamino)-2-methoxybenzoate (EL2), 2-(dibutylamino)ethyl 4-(butylamino)-5-chloro-2-methoxybenzoate (EL3), 2-(diethylamino)ethyl 4-(butylamino)-3-methoxybenzoate (EL4), 2-(dimethylamino)ethyl 4-(butylamino)-2-methoxybenzoate (EL5), 4-(butylamino)-3-methoxybenzoic acid (EL6), 2-(dimethylamino)ethyl 4-(butylamino)-3-methylbenzoate (EL7), 2-(diethylamino)ethyl 4-(butylamino)-3-methylbenzoate (EL8), and 2-(diethylamino)ethyl 4-(butylamino)-5-chloro-2-methoxybenzoate (EL9). In some aspects, the Ryanodine receptor antagonist is tetracaine. The tetracaine can be in substantially in the deprotonated base form as tetracaine base. In some aspects, the tetracaine base is the sole active ingredient in the composition.

In some aspects, the present invention relates to Ryanodine receptor antagonist compositions which can include PEG400 (polyethylene glycol, mean average molecular weight of 400). In some aspects, the Ryanodine receptor antagonist compositions of this disclosure include only non-aqueous components (e.g., they do not include an aqueous component). In some aspects, the present invention relates to Ryanodine receptor antagonist compositions which comprise from about 2 to about 8 wt. % tetracaine, from about 4 to about 6 wt. % tetracaine, or about 6 wt. % tetracaine. In some aspects, the present invention relates to compositions which can further comprise a non-aqueous vehicle.

In some aspects, the present invention relates to the use of a Ryanodine receptor antagonist in the preparation of a medicament for treating HSV-2 associated genital herpes in a subject. In some aspects, the Ryanodine receptor antagonist is tetracaine base. In some aspects, the tetracaine base is in a non-aqueous formulation.

### BRIEF DESCRIPTION OF DRAWINGS

The drawings form part of the present specification and are included to further demonstrate certain aspects of the embodiments described herein. These embodiments may be better understood by reference to one or more of the following drawings in combination with the detailed description.

FIGS. 1A-1C shows neutralization of Acyclovir in different MOIs of HSV-2 in HEK293 RyR1 cells on day 3 with (FIG. 1A) MOI=0.01, (FIG. 1B) MOI=0.005 and (FIG. 1C) MOI=0.001. Data was normalized to the average absorbance of the virus-only wells, considered to be 0% inhibition, to calculate % inhibition values. The % inhibition values were graphed using a sigmoidal dose response model and IC50 values were derived from the inhibition curves.

FIGS. 2A-2B shows Neutralization of Acyclovir of HSV-2 in different seeding densities of HEK293 RyR1 cells on day 3 with seeding density of (FIG. 2A) 5E4 cells/well and (FIG. 2B) 8E4 cells/well. Data was normalized to the average absorbance of the virus only wells, considered to be 0% inhibition, to calculate % inhibition values. The % inhibition values were graphed using a sigmoidal dose response model and IC50 values were derived from the inhibition curves.

FIGS. 3A-3D shows neutralization of high starting concentration of Acyclovir of two different MOIs of HSV-2 in different seeding densities of HEK293 RyR1 cells on day 3. FIG. 3A shows neutralization of acyclovir against 0.01 MOI HSV-2 in 3E4 cells/well of HEK-293 RyR1 cells on day 3. FIG. 3B shows neutralization of acyclovir against 0.01 MOI HSV-2 in 5E4 cells/well of HEK-293 RyR1 cells on day 3. FIG. 3C shows neutralization of acyclovir against 0.02 MOI HSV-2 in 3E4 cells/well of HEK-293 RyR1 cells on day 3. FIG. 3D shows neutralization of acyclovir against 0.02 MOI HSV-2 in 5E4 cells/well of HEK-293 RyR1 cells on day 3. Data was normalized to the average absorbance of the virus-only wells, considered to be 0% inhibition, to calculate % inhibition values. The % inhibition values were graphed using a sigmoidal dose response model and IC50 values were derived from the inhibition curves.

FIGS. 4A-4B shows neutralization of Acyclovir and Tetracaine against HSV-2 in HEK293 RyR1 cells on day 3. FIG. 4A shows neutralization of acyclovir against 0.01 MOI HSV-2 in 5E4 cells/well of HEK-293 RyR1 cells on day 3. FIG. 4B shows neutralization of tetracaine against 0.01 MOI HSV-2 in 5E4 cells/well of HEK-293 RyR1 cells on day 3.

FIGS. 5A-5D shows cell morphology of cells following tetracaine incubation and virus infection on day 3. FIG. 5A shows cells only. FIG. 5B shows virus only. FIG. 5C shows 100 μg/ml acyclovir-treated cells. FIG. 5D shows 100 mg/ml tetracaine treated cells.

FIGS. 6A-6B shows Calcium flux assay with the Fluo-4 Direct Calcium assay kit. ΔRFU is given in relative fluorescent units (RFU) as the maximum response (tetracaine or virus treated cells) minus the minimum response (cells only). FIG. 6A shows cyclovir-treated cells. FIG. 6B shows tetracaine treated cells. Both treatments demonstrate calcium flux is significantly reduced by the administration of a RyR1 antagonist, acyclovir or tetracaine, with tetracaine showing a significantly larger reduction (4000 RFU drop for tetracaine compared to about 2000 RFU drop for acyclovir). Tetracaine is therefore a stronger reducer of calcium flux than acyclovir.

### DETAILED DESCRIPTION

The inventions relate to the treatment of HSV-2 in a subject, reduction of HSV-2 genital ulcers on a subject, and the killing of HSV-2 viral pathogens by the use of compositions comprising one or more Ryanodine receptor antagonists. The compositions of this disclosure can comprise tetracaine, in particular tetracaine base, as a representative Ryanodine receptor antagonist.

Without being bound by theory, herpes simplex viruses (HSV) are the leading cause of genital ulcers. Viral entry is a multicomponent process that requires multiple interactions at the cell surface involving four envelope glycoproteins, gD, gB, and heterodimers of gH-gL. gB plays the dominant role in mediating binding for HSV-2. After engagement of this attachment receptor, gD interacts with one of several coreceptors. Independently of its role in attachment, gB is also required for fusion as are heterodimers of gH-gL. The concentration of intracellular free Ca²⁺ ([Ca²⁺]ᵢ) regulates a variety of cellular processes. The specificity of Ca²⁺ responses is characterized by the frequency, amplitude, duration, and spatial restriction of the Ca²⁺ signaling (Berridge M., (2005). Unlocking the secrets of cell signaling. Annu. Rev. Physiol 67, 1-21). Intracellular Ca²⁺ signals can be localized to domains at or just below the plasma membrane (referred to as membrane Ca²⁺), or they may be associated with release from the endoplasmic reticulum (ER) and mitochondria. HSV-1 and HSV-2 trigger the release of Ca²⁺ and that Ca²⁺ signaling plays an important role in viral entry (Cheshenko, et al., (2003). Herpes simplex virus triggers activation of calcium-signaling pathways. J. Cell Biol 163, 283-293). Pharmacological inhibition of, or chelation of, intracellular Ca²⁺ can prevented viral infection. HSV may hijack Ca²⁺ signaling pathways to trigger entry. (Cheshenko, et al., Molecular Biology of the Cell, 18 (8), 3119-3130, August 2007).

Intracellular Ca²⁺ is an important secondary messenger for signal transduction and is essential for cellular processes. RyRs are large conductance channels capable of creating rapid transient increases of cytosolic Ca²⁺. RyRs exist in three isoforms (RyR 1-3) and derive their nomenclature from the plant alkaloid ryanodine, which binds to RyRs with high affinity and specificity and displays preferential interactions with the open state of the channel allowing its usage to evaluate the functional state of the channel. RyRs are modulated directly or indirectly by the dihydropyridine receptor (DHPR; also known as L-type Ca²⁺ channel, Cav1.1/1.2) and by various ions, small molecules and proteins, e.g., Ca²⁺, Mg²⁺, protein kinase A (PKA), FK506 binding proteins (FKBP12 and 12.6), calmodulin (CaM), Ca²⁺/calmodulin-dependent protein kinase II (CaMKII), calsequestrin (CSQ), triadin, junction, and the small molecules of this disclosure (e.g., tetracaine). RyRs are involved in signal transduction in the nervous system and in osteoclasts where they contribute to secretion, synaptic plasticity, learning, and apoptosis. The inventors recognized that RyRs are also involved in the proliferation of HSV, including HSV-2, and that inhibiting and/or antagonizing RyRs is an effective mechanism to kill HSV-2 viral pathogens, reduce the number of genetic ulcers arising from HSV-2, and/or treating HSV-2 in a subject having such.

The inventors discovery includes that tetracaine can induce closures in RyRs. Luminal and cytoplasmic tetracaine induces closed events. The binding rate decreases with increasing RyR open probability, consistent with a closed state mechanism with no detectable binding to open channels. At pH 7.4, its voltage-dependence points to a cation binding site within the bilayer, near its luminal interface. At pH 9.5, the slow mechanism is independent of voltage, indicating that it also responds to neutral tetracaine molecules. Cytoplasmic and luminal tetracaine induce brief closures. It is insensitive to the open state of the RyR, independent of voltage and markedly diminished at pH 9.5 indicating that it is caused exclusively by tetracaine cations binding outside the transmembrane electric field. The action of trans-membrane pH gradients on the fast mechanism points to a cytoplasmic location for the binding site. Cytoplasmic tetracaine reduces the ionic conductance of the channel at mM concentrations. The slow mechanism (IC50 of −200 mM) is a more potent form of block than the fast mechanism or conduction block (IC50˜2 mM) and these mechanisms can explain the differences in the tetracaine inhibition seen at low and high concentrations, or at neutral and basic pH conditions. Thus, the inventors recognized that presenting tetracaine in base form would be more potent as a Ryanodine receptor (RyR) antagonist.

Tetracaine (2-(dimethylamino)ethyl 4-(butylamino)benzoate) can exists in both a base form and a protonated (or salt) form. The base form is soluble in hydrophobic media, and the salt form soluble in alcohol and aqueous solutions. Tetracaine as a base fluxes more readily than the salt form through the dermis (K. J. Miller, Yet al., Solubility and in vitro percutaneous absorption of tetracaine from solvents of propylene glycol and saline, International Journal of Pharmaceutics, 98 (1-3), Pages 101-111 (1993), doi.org/10.1016/0378-5173(93)90046-I). Tetracaine salt forms micelles wherein the hydrophobic n-butyl chain overlaps with the chains of other tetracaine compounds.

### Mechanism of Action of Tetracaine on Inhibiting HSV-2 Replication by Binding to Ryanodine Receptors

Intercellular calcium (Ca2+) has a significant impact on the herpes virus. The virus uses Ca2+ to facilitate its entry, replication, and assembly.

Virus entry: Herpes viruses need to enter the host cell in order to replicate. They do this by binding to receptors on the cell surface and then fusing their envelope with the cell membrane. This fusion process is triggered by a rise in intracellular Ca2+ levels.

Viral gene expression: Once the virus has entered the cell, it needs to express its genes in order to replicate. This process is also regulated by Ca2+. For example, the herpes simplex virus (HSV) uses Ca2+ to activate its transcription factors, which are proteins that bind to DNA and initiate transcription of viral genes.

Viral protein synthesis: The herpes virus also needs to synthesize proteins in order to replicate. This process is also regulated by Ca2+. For example, the HSV uses Ca2+ to activate its RNA polymerase, which is an enzyme that copies the viral RNA genome into mRNA.

Virion assembly: Once the viral genes have been expressed and the proteins have been synthesized, the virus needs to assemble into virions (virus particles). This process is also regulated by Ca2+. For example, the HSV uses Ca2+ to help package its DNA genome into the capsid, which is the protein shell that protects the genome.

Virion release: Finally, the herpes virus needs to release the virions from the host cell in order to spread. This process is also regulated by Ca2+. For example, the HSV uses Ca2+ to activate its matrix protein, which helps to release the virus from the cell.

Overall, Ca2+ is a critical molecule for the herpes virus. By hijacking the host cell's Ca2+ signaling pathways, the virus can ensure that it has the Ca2+ it needs to replicate and spread.

Tetracaine affects intercellular calcium (Ca2+) levels. In particular, it inhibits the release of Ca2+ from intracellular stores because tetracaine binds to ryanodine receptors, which are proteins that control the release of Ca2+ from these stores.

The inhibition of Ca2+ release by tetracaine can have a number of effects on cells which can include or exclude: reduce the contraction of muscle cells, inhibit the release of neurotransmitters, and block the growth of cancer cells.

In the context of intercellular Ca2+ levels, the inhibition of Ca2+ release by tetracaine can lead to a decrease in the overall levels of Ca2+ in the cells. This can have a number of effects on cell signaling and function.

Ryanodine receptors (RyRs) are proteins that are found in the endoplasmic reticulum (ER) of cells. They are involved in the release of calcium ions from the ER into the cytoplasm.

The herpes virus hijacks RyRs to its advantage. The virus produces a protein called v-Jun N-terminal protein kinase (v-JNK), which binds to and activates RyRs. This leads to the release of calcium ions from the ER, which in turn activates a number of cellular processes that promote viral replication.

For example, the release of calcium ions activates transcription factors, which are proteins that bind to DNA and initiate transcription of viral genes. The release of calcium ions also activates protein kinases, which are enzymes that phosphorylate other proteins, changing their activity.

The activation of transcription factors and protein kinases leads to the production of viral proteins and the assembly of viral particles. The release of calcium ions also contributes to the lysis of infected cells, which releases the virus into the environment.

Overall, RyRs play a critical role in herpes virus replication. By hijacking RyRs, the virus can ensure that it has the calcium ions it needs to replicate and spread.

RyRs are involved in herpes virus replication by the following mechanisms:
1. Activation of transcription factors: The release of calcium ions from the ER activates transcription factors, which are proteins that bind to DNA and initiate transcription of viral genes. This is essential for the production of viral proteins.
2. Activation of protein kinases: The release of calcium ions from the ER activates protein kinases, which are enzymes that phosphorylate other proteins, changing their activity. This is important for a number of cellular processes, including the assembly of viral particles and the lysis of infected cells.
3. Lysis of infected cells: The release of calcium ions from the ER can contribute to the lysis of infected cells. This releases the virus into the environment, allowing it to infect other cells.

The tetracaine compositions of this disclosure leaves the intercellular pH near 8, which is an alkalosis state. In alkalosis, the calcium channels are less likely to be open, which reduces the amount of calcium ions that enter the cell.

Tetracaine is known to protect keratinocytes from damage caused by ultraviolet radiation (UVR). Tetracaine increases the production of the antioxidant glutathione, which helps to protect cells from ROS. ROS, however, are involved in the pathogenesis of herpes virus infections. ROS can contribute to the spread of the HSV-2 virus, the development of symptoms, and the long-term effects of the infection.

Glutathione is a powerful antioxidant that can help protect cells from damage. It is also involved in a number of other cellular processes, including DNA repair and immune function.

The exact mechanism by which glutathione exerts its antiviral effects is not fully understood. However, it is thought that glutathione may help to scavenge reactive oxygen species (ROS), which can damage cells and promote viral replication. Glutathione may also help to repair DNA damage caused by the virus.

More research is needed to fully understand the role of glutathione in herpes virus infection. However, the available evidence suggests that glutathione may be a potential antiviral agent.

Glutathione can inhibit the herpes virus by: scavenge ROS, repair DNA damage, inhibit the production of viral proteins, inhibit the assembly of viral particles, and/or induce apoptosis of infected cells.

In summary, the primary impact of representative tetracaine compositions of this disclosure is the direct binding of its active ingredient Tetracaine (base form) to ryanodine receptors in the cell wall which shuts down Ca(2+) release, which in turn shuts down the ability of the virus to reproduce, bind, and spread.

The inventors have surprisingly found that topical administration of a Ryanodine receptor antagonist (e.g., tetracaine) composition is effective, and fast, in the treatment in a human subject of genital herpes originating from HSV-2. The base form of tetracaine can be stabilized by maintaining the compound in a non-aqueous media because the tetracaine base is more soluble in the non-aqueous media thereby driving the solubility equilibrium into the base form.

### Definitions

As used herein, the term “about” refers to the stated value and typical degrees of error of the measurement of said value. If not otherwise explicitly recited herein, the term “about” refers to any number within +/−10% of the recited value.

The term “and/or” as used herein is to be taken as specific disclosure of each of the two specified features or components with or without the other. For example “A and/or B” is to be taken as specific disclosure of each of (i) A, (ii) B and (iii) A and B, just as if each is set out individually herein.

An “antagonist” or “inhibitor” may be a polypeptide, nucleic acid, carbohydrate, lipid, small molecule, an oligonucleotide, an oligopeptide, RNA interference (RNAi), antisense, a recombinant protein, an antibody, or fragments thereof or conjugates or fusion proteins thereof, which inhibits the activity of a selected target. The term “inhibits” when referring to a targeted binding agent, such as a small molecule (e.g., tetracaine as a Ryanodine receptor antagonist), relates to the ability of said agent to eliminate, reduce, or significantly reduce, the activity of a target. “Inhibiting” the biological activity of a selected target means an inhibition of the target activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% in comparison with the biological activity in the absence of a antagonist of the invention.

As used herein, the terms “subject”, or “patient” may mean either a human or non-human animal. The term includes, but is not limited to, mammals (e.g., humans, other primates, pigs, rodents (e.g., mice and rats or hamsters), rabbits, guinea pigs, cows, horses, cats, dogs, sheep, and goats). In certain embodiments, the subject is a human.

As used herein, the term “treatment” (and grammatical variations thereof such as “treat” or “treating”), refers to clinical intervention in an attempt to alter the natural course of the individual, tissue or cell being treated, and can be performed either for prophylaxis or during the course of clinical pathology. Desirable effects of treatment include, but are not limited to, preventing occurrence or recurrence of a disease, disorder or condition, alleviation of signs or symptoms, diminishment of any direct or indirect pathological consequences of the disease, decreasing the rate of disease progression, amelioration or palliation of the disease state, and remission or improved prognosis. In some embodiments, compounds, methods and compositions of the invention can be used to delay development of a disease, disorder or condition, or to slow the progression of a disease, disorder or condition. The term does not necessarily imply that a subject is treated until total recovery. Accordingly, “treatment” includes reducing, alleviating or ameliorating the symptoms or severity of a particular disease, disorder or condition or preventing or otherwise reducing the risk of developing a particular disease, disorder or condition. It may also include maintaining or promoting a complete or partial state of remission of a condition.

As used herein, the terms “prevent,” “preventing,” and “prevention” as used herein means the prevention of a disease in a mammal, e.g., in a human, including (a) avoiding or precluding the disease; (b) affecting the predisposition toward the disease; (c) preventing or delaying the onset of at least one symptom of the disease.

As used herein, the “small molecule” means to have a molecular weight below about 2000 daltons, and is generally an organic compound. A small molecule can be an active agent of a prodrug.

As used herein, “effective amount” refers to an amount effective, at dosages and for periods of time necessary, to achieve the desired therapeutic or prophylactic result. For example, and not by way of limitation, an “effective amount” can refer to an amount of a compound or composition, disclosed herein, that is able to treat the signs and/or symptoms of a disease, disorder or condition.

As used herein, “therapeutically effective amount” of a substance/molecule of the invention, agonist or antagonist may vary according to factors such as the disease state, age, sex, and weight of the individual, and the ability of the substance/molecule, agonist or antagonist to elicit a desired response in the individual. A therapeutically effective amount is preferably also one in which any toxic or detrimental effects of the substance/molecule, agonist or antagonist may be outweighed by the therapeutically beneficial effects.

As used herein, “prophylactically effective amount” refers to an amount effective, at dosages and for periods of time necessary, to achieve a desired prophylactic result. Typically but not necessarily, since a prophylactic dose is used in subjects prior to or at an earlier stage of a disease, disorder or condition, the prophylactically effective amount will be less than the therapeutically effective amount.

The term “pharmaceutical formulation” refers to a preparation which is in such form as to permit the biological activity of an active ingredient contained therein to be effective, and which does not contain additional components that are unacceptably toxic to a subject to which the formulation would be administered.

A “pharmaceutically acceptable carrier,” as used herein, refers to an ingredient in a pharmaceutical formulation, other than an active ingredient, which can be safely administered to a subject. A pharmaceutically acceptable carrier includes, but is not limited to, a buffer, excipient, stabilizer, or preservative.

### Compositions

In some embodiments, the Ryanodine receptor antagonist formulations of this disclosure can comprise a carrier vehicle. The carrier vehicle can further include or exclude: viscosity modifiers, fragrants, colorants, skin penetrating enhancers, stabilizers, preservatives, tonicity agents, solubilizers, and surfactants. While the range of tetracaine can be from 2 to 8 wt. % of the total formulation, the range of the other components may vary, and if selected, can be at any range up to the total composition weight.

In some embodiments, the formulation can include one or more skin penetration agents. The skin penetration agent can be a polyethyleneglycol, polypropylene glycol, oleic acid, emu oil, laurocapram, Monoolein, Oxazolidinones, SEPA (2-n-nonyl-1,3-dioxolane), Polyoxyethylene sorbitan monopalmitate, and d-limonene. In some embodiments, the polyethyleneglycol (PEG) can include or exclude: PolyOx WSR 301 (PEG90) and PEG400 USP.

In some embodiments, the formulation can include a hydrophobic carrier. The hydrophobic carrier can be a topical ointment base. The topical ointment base can be Jelene (95% mineral oil and 5% low molecular weight polyethylene), Vaseline (white petrolatum), or lanolin.

In some embodiments, the formulation can include a viscosity enhancer to increase the residence time of the formulation on the genital ulcer site. As a non-limiting example, Sodium Carboxymethylcellulose can be used as a viscosity enhance for the present invention.

In some embodiments, essential oils can be added to the compositions containing Ryanodine receptor antagonist(s) as fragrance or aromatic agents, and/or as additional antimicrobial agents. Examples of essential oils useful in the compositions described herein can include or exclude, thymol, menthol, sandalwood, camphor, cardamom, cinnamon, jasmine, lavender, geranium, juniper, menthol, pine, lemon, rose, eucalyptus, clove, orange, oregano, chocolate mint, linalool, spearmint, peppermint, lemongrass, bergamot, citronella, cypress, nutmeg, spruce, tea tree, wintergreen (methyl salicylate), vanilla, and the like. In some embodiments, the essential oils can be selected from thymol, sandalwood oil, wintergreen oil, eucalyptol, pine oil, and combinations thereof. In some embodiments, essential oils can be present in the compositions in an amount ranging from 0% to 5 wt % based on the total weight of the composition. In some embodiments, essential oils can be present in the composition in an amount of at least 0.1 wt %, or at least 0.25 wt %, or at least 0.5 wt %, based on the weight of the composition. In some embodiments, the compositions of the invention can include or exclude a terpene. The terpene can include or exclude myrcene, pinene, caryophyllene, geraniol, sabinene, d-limonene, nerol, Citronellol, Lavandulol, Terpineol, Farnesol, Nerolidol, bisabolol, cubebol, and retinol.

In some embodiments, the formulations can include or exclude a surfactant. Surfactants (surface active agents) or solubilizing agents suitable for the present invention are those acceptable for use in topical preparations. The surfactants can be ionic or non-ionic. Preferably, the surfactant is non-ionic. Useful surfactants include but are not limited to polysorbate 80, polyoxyl stearates, tyloxapol, polyethoxylated castor oils, poloxamers, polaxamines, medium and long chain fatty acids and phospholipids. The concentration of the surfactant in the formulation is about 0.01-3%, preferably 0.01-2%, more preferably 0.1-1% w/v. In some embodiments, when a surfactant is included, the surfactant does not exhibit an anti-HSV-2 activity. In some embodiments, the formulations of this invention do not include a surfactant.

The pharmaceutical formulations of this invention may further comprise one or more pharmaceutically acceptable excipients.

The Ryanodine receptor inhibitor compounds of this disclosure may be present in the formulation in a substantially isolated form. It will be understood that the product may be mixed with carriers or diluents that will not interfere with the intended purpose of the product and still be regarded as substantially isolated. A product of the invention may also be in a substantially purified form, in which case it will generally comprise about 80%, 85%, or 90%, e.g. at least about 88%, at least about 90, 95 or 98%, or at least about 99% of a compound, or dry mass of the preparation.

Pharmaceutically acceptable diluents, carriers and/or excipients include those suitable for veterinary use as well as human pharmaceutical use. By way of example, diluents, carriers and/or excipients include solutions, solvents, dispersion media, delay agents, polymeric and lipidic agents, and the like.

Suitable carriers and diluents include non-aqueous components which can include or exclude: dextrose, glycerol, propylene glycol, C1-C18 monofunctional alcohols, C1-C18 symmetrical alcohols, dimethyl isosorbide, and combinations thereof. In some embodiments the pharmaceutically acceptable carrier or diluent may be or contain a thermosetting poloxamer (which may be a liquid or gel, depending on the temperature), a carboxycellulose (e.g. carboxymethylcellulose), a collagen (e.g., a Type I collagen), a collagenous material comprising tropocollagen, a hyaluronan or derived-hyaluronic acid, and/or an oil (e.g., Emu oil).

Compositions may take the form of any standard known dosage form including tablets, pills, capsules, semisolids, powders, sustained release formulation, solutions, suspensions, elixirs, aerosols, liquids for injection, gels, creams, transdermal delivery devices (for example, a transdermal patch), inserts such as ocular inserts, or any other appropriate compositions.

Preferably the Ryanodine receptor modulator compound is combined with a pharmaceutically acceptable carrier or diluent to produce a pharmaceutical composition.

The pharmaceutical compositions may be formulated in accordance with standard techniques known in the art, including those as may be found in such standard references as Gennaro AR: Remington: The Science and Practice of Pharmacy, 20th ed., Lippincott, Williams & Wilkins, 2000, for example.

The Ryanidine receptor modulating compounds may also be admixed, conjugated or otherwise associated with other molecules, molecule structures or mixtures of compounds, as for example, liposomes, receptor-targeted molecules, or other formulations, for assisting in uptake, distribution and/or absorption.

In some embodiments, the surfactant is a lipid. Preferred lipids include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

In some embodiments, the formulations of the inventions include a tonicity agent. The tonicity agent is present in an amount to achieve a final formulation tonicity between 220-360 mOsm/kG, preferably 250-340 mOsm/kG, and most preferably between 260 and 320. The tonicity agent can be ionic or non-ionic. Non-ionic tonicity agents include compounds comprising 1,2-diols, such as glycerol, mannitol, erythritol; and sugars such as dextrose. Other non-ionic tonicity agents which also function as cosolvents can also be used such as polyethylene glycol and propylene glycol. The non-ionic tonicity agent can be present in an amount of 0-20%, preferably 0-10%, more preferably 0-5%. The non-ionic agents can be selected from: glycerol, mannitol and dextrose, in an amount 2-6%.

In some embodiments, the formulations can comprise a preservative. Suitable preservatives include benzyl alcohol, methyl parabens, propyl parabens, borate, chlorobutanol, and benzethonium chlorides. Typically, such preservatives are employed at a level of from 0.001-1%, preferably, 0.001-0.25%, and most preferably 0.001-0.2%.

### Ryanodine Receptor Antagonists

Ryanodine receptors (i.e., RyRs), which have been considered as the largest intracellular calcium ion release channels known to date, located in the sarcoplasmic reticulum/endoplasmic reticulum (SR/ER), are necessary for calcium ion release from intracellular stores to the cytoplasm. They are homotetramers with each monomer containing nearly 5000 amino acid residues. There exist three isoforms of RyRs in the mammalian, where RyR1 is mainly expressed in the skeletal muscles, RyR2 is the predominant isoform in the cardiac muscles, and RyR3 is initially identified in the brain.

In some embodiments, the Ryanodine receptor antagonist can be selected from: Tetracaine, procaine, Dantrolene, Chlorantraniliprole, cyantraniliprole, flubendiamide, cyclaniliprole, tetraniliprole, Ryanodine, JTV 519 fumarate ((4-[3(1-(4-benzyl)piperidinyl)propionyl]-7-methoxy-2,2,4,5-tetrahydro-1,4-benzothiazepine, fumarate salt), Ruthenium Red, DHBP (1,1′-diheptyl-4,4′-bipyridium), VK-II-86, Phenytoin (diphenylhydantoin), Flecainide, Carvedilol, EL20 (2-(diethylamino)ethyl 4-(butylamino)-2-methoxybenzoate), Xanthotoxol, 5-hydroxy-1,4-naphthalenedione, Rycal (ARM210) (Benzoic acid, 4-((2,3-dihydro-7-methoxy-1,4-benzothiazepin-4(5H)-yl)methyl)-), S107 (2,3,4,5-tetrahydro-7-methoxy-4-methyl-1,4-benzothiazepine), 4-(2-aminopropyl)-3,5-dichloro-N,N-dimethylaniline (FLA 365), 2-(dimethylamino)ethyl 4-(butylamino)-5-chloro-2-methoxybenzoate (EL1), 2-(dibutylamino)ethyl 4-(butylamino)-2-methoxybenzoate (EL2), 2-(dibutylamino)ethyl 4-(butylamino)-5-chloro-2-methoxybenzoate (EL3), 2-(diethylamino)ethyl 4-(butylamino)-3-methoxybenzoate (EL4), 2-(dimethylamino)ethyl 4-(butylamino)-2-methoxybenzoate (EL5), 4-(butylamino)-3-methoxybenzoic acid (EL6), 2-(dimethylamino)ethyl 4-(butylamino)-3-methylbenzoate (EL7), 2-(diethylamino)ethyl 4-(butylamino)-3-methylbenzoate (EL8), 2-(diethylamino)ethyl 4-(butylamino)-5-chloro-2-methoxybenzoate (EL9), or combinations thereof.

In some embodiments, the Ryanodine receptor antagonist can be selected from: the 1,4-Benzothiazepines as described in U.S. Pat. No. 8,853,198B2, U.S. Ser. No. 11/717,526B2, U.S. Ser. No. 11/504,383B2, US20230399324A1, U.S. Ser. No. 11/905,271B2, each of which is herein incorporated by reference.

In some embodiments, the Ryanodine receptor antagonist can be selected from those described in Li et al. (Treatment of catecholaminergic polymorphic ventricular tachycardia in mice using novel RyR2-modifying drugs. Int J Cardiol. 2017 Jan. 15; 227:668-673. doi: 10.1016/j.ijcard.2016.10.078. Epub 2016 Oct 29. PMID: 27838126), herein incorporated by reference. The Ryanodine receptor antagonist can be EL1, EL2, EL3, EL4, EL5, EL6, EL7, EL8, or EL9:

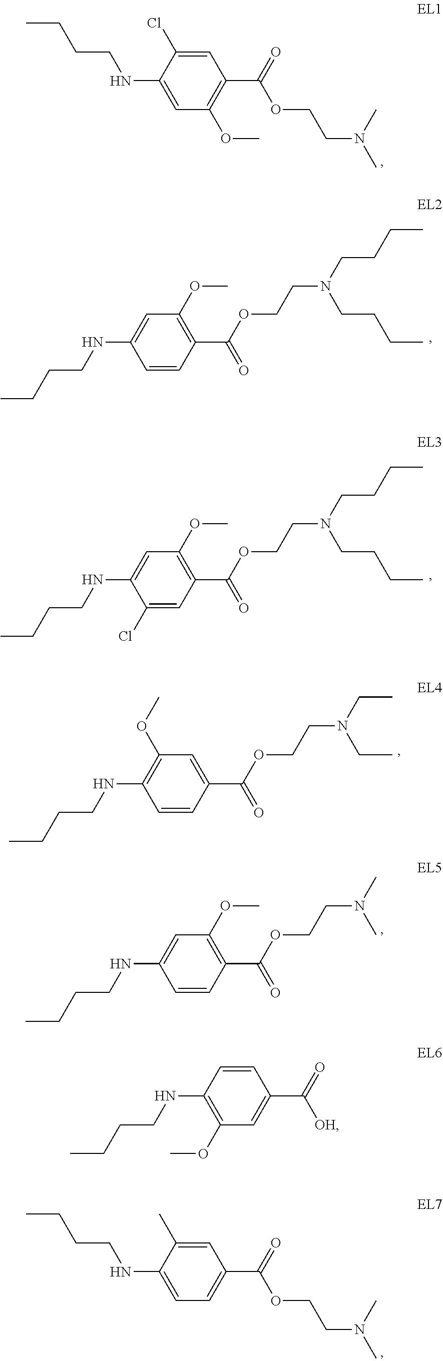

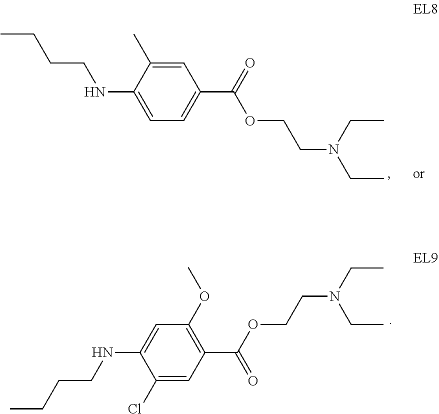

Tetracaine comprises a substituted amine moiety and thus has the unique property that it is soluble in hydrophobic media when in base form, and relatively soluble in hydrophilic media when in salt (ionized) form. The inventors, having recognized that the Ca2+ channel inhibiting activity is pH-dependent, have formulated tetracaine to be in base form using the compositions described herein. By excluding exogenous water from the compositions, the compositions of the present invention comprise tetracaine primarily in base form, which is the more potent form of tetracaine (compared to its salt form), and is also more easily passed through the dermis while in base form. Thus, in a preferred embodiment, the compositions of this disclosure are hydrophobic, thereby shifting the equilibrium of the tetracaine to the deprotonated (e.g., base) form.

### Combination of Ryanodine Receptor Antagonists

In some embodiments, provided are methods of treating a disease, disorder, or condition. The methods may comprise administering to a subject a combination of Ryanodine receptor antagonists. The combination of Ryanodine receptor antagonists can be administered as separate, or different formulations. In some embodiments, a first Ryanodine receptor antagonist can be administered topically, and a second Ryanodine receptor antagonist can be administered systemically. In some embodiments, the first and second Ryanodine receptor antagonists can be within the same formulation. In some embodiments, the first and second Ryanodine receptor antagonists can be administered topically but at different times.

For the separate administration (coadministration) of the first Ryanodine receptor antagonist and the second Ryanodine receptor antagonist (e.g. tetracaine), the administrations can be sequential or simultaneous. For sequential administrations, the first Ryanodine receptor antagonist and the second Ryanodine receptor antagonist (e.g. tetracaine) can be administered within one hour of each other, within one day of each other, within one week of each other, or within one month of each other. For simultaneous administration (coadministration), the first Ryanodine receptor antagonist and the second Ryanodine receptor antagonist (e.g. tetracaine) can be administered together as a mixture, or as a formulation comprising both the first Ryanodine receptor antagonist and the second Ryanodine receptor antagonist (e.g. tetracaine).

### Certain Methods

In some embodiments, the invention provides methods of killing a HSV-2 viral pathogen, the methods comprising contacting a genital ulcer on the subject with a Ryanodine receptor antagonist, in an amount effective to modulate HSV-2 activity, and contacting a genital ulcer on the subject with a Ryanodine receptor antagonist, for example, in an amount effective to modulate HSV-2 activity.

In some embodiments, the invention provides methods of treating HSV-2 in a subject having HSV-2, the methods comprising contacting a genital ulcer on the subject with a Ryanodine receptor antagonist, in an amount effective to treat the subject.

In some embodiments, the invention provides methods of reducing the number of genital ulcers arising from HSV-2, the methods comprising contacting a genital ulcer on the subject with a Ryanodine receptor antagonist, in an amount effective to heal the genital ulcer.

For separate or common administration, the dosage unit of the Ryanodine receptor antagonist is of a formulation ranging from about 2 to about 8 wt. % of the Ryanodine receptor antagonist (e.g., tetracaine). In some embodiments, the dosage unit of the Ryanodine receptor antagonist is of a formulation ranging from about 4 to about 6 wt. % Ryanodine receptor antagonist (e.g., tetracaine). In some embodiments, the dosage unit of the Ryanodine receptor antagonist is of a formulation comprising about 6 wt. % of the Ryanodine receptor antagonist (e.g., tetracaine).

The dosage unit of the compositions of this invention can, in some embodiments, be a total of about 100 mg, 200 mg, 300 mg, 400 mg, 500 mg, 600 mg, 700 mg, 800 mg, 900 mg, 1 g, 2 g, 3 g, 4 g, 5 g, or any value between the aforementioned amounts.

In certain embodiments, the Ryanodine receptor antagonist (e.g., tetracaine) may be administered at about 2 wt. % to about up to 8 wt. % of the total amount administered to the genital ulcer site on the subject having or suspected of having HSV-2. In one embodiment, the Ryanodine receptor antagonist (e.g., tetracaine) composition is applied at greater than about 2 wt. %. Preferably, the Ryanodine receptor antagonist (e.g., tetracaine) composition is applied at about 6 wt. % final concentration. The inventors have recognized that tetracaine compositions higher than 8 wt. % may cause skin irritation, thereby defeating the purpose of using tetracaine as a therapeutic agent. Concentrations lower than 6 wt. % can be used for more skin-sensitive subjects, or for longer-duration formulations (e.g., when impregnated into a bandage which is contacted to the genital ulcer on the subject).

It should be appreciated that administration may include a single daily dose, administration of a number of discrete divided doses, or continuous administration, as may be appropriate. By way of example, unit doses may be administered once or more than once per day, for example 1, 2, 3, 4, 5 or 6 times a day to achieve a desired total daily dose. In some embodiments, the dosage unit of the compositions of this invention can be administered from one to twenty times per day.

For separate or common administration, the formulation may be prepared to provide for rapid or slow release; immediate, delayed, timed, or sustained release; or a combination thereof. Formulations may be in the form of liquids, solutions, suspensions, emulsions, elixirs, syrups, electuaries, drops (including but not limited to eye drops), tablets, granules, powders, lozenges, pastilles, capsules, gels, ointments, creams, lotions, oils, foams, sprays, mists, or aerosols. As an additional embodiment, the pharmaceutical formulation can be contained within, delivered by, or attached to a bandage which is applied to the surface of a genital ulcer arising from HSV-2.

In some embodiments, this disclosure provides for use of a Ryanodine receptor antagonist in the preparation of a medicament for treating HSV-2, reducing the number of blisters arising from infection from HSV-2, or ameliorating the pain or symptoms associated with HSV-2 infection.

### Manufacture and Stability

The Ryanodine receptor antagonists of this invention can commercially obtained. In one aspect, the formulations of this invention will comprise a non-aqueous formulation of the Ryanodine receptor antagonist. In one embodiment, the non-aqueous (e.g., hydrophobic) media shifts the equilibrium balance of a tetracaine compound into the base form, the more potent form of the compound for modulating RyRs activity, thereby acting as a more potent treatment of HSV-2.

In some embodiments, the formulations of this invention are substantially pure. By substantially pure is meant that the formulations comprise less than about 10%, 5%, or 1%, and preferably less than about 0.1%, of any compound impurity. In some embodiments the total impurities, including metabolities of the Ryanodine receptor antagonist, will be not more than 15%. In some embodiments the total impurities, including metabolities of the Ryanodine receptor antagonist, will be not more than 12%. In some embodiments the total impurities, including metabolities of the Ryanodine receptor antagonist, will be not more than 11%. In other embodiments the total impurities, including metabolities of Ryanodine receptor antagonist, will be not more than 10%.

In some embodiments, the purity of the formulations of this invention may be measured using a method selected from anion exchange HPLC (AEX-HPLC) or mass spectrometry. Mass spectrometry may include LC/MS, or LC/MS/MS. The assay may in some embodiments comprise both AEX-HPLC and LC/MS.

Sterile compositions comprising the Ryanodine receptor antagonists of this invention prepared using aseptic processing by dissolving the Ryanodine receptor antagonist in the formulation vehicle. In one embodiment, the formulation may also be sterilized by filtration. Excipients used in the manufacture of the formulations of this invention are widely used in pharmaceutical products and released to pharmacopeial standards.

### EXAMPLES

### Example 1. Tetracaine Composition

As a representative embodiment of this disclosure, a topical composition comprising 6 wt. % tetracaine base having the formulation shown in the table below was prepared by mixing the appropriate components in the specified amounts.

| | WEIGHT |
|---|---|
| INGREDIENT | (% w/w) |
| Tetracaine USP Base | 6.0 |
| Sodium Carboxymethylcellulose USP, medium viscosity | 4.0 |
| PolyOx WSR 301 | 5.0 |
| PEG400 (Polyethylene glycol) USP | 10.0 |
| Plasticized Base (Jelene) (95% mineral oil and 5% low | 74.33 |
| molecular weight polyethylene) | |
| Peppermint Oil, NF | 0.67% |

The topical composition can be prepared as follows. Sodium carboxymethyl cellulose, PolyOx WSR 301, and tetracaine are ground in separate glass mortars to a fine powder and set aside. Ten percent extra tetracaine is weighed out because some of the tetracaine may adhere to the mortar. By starting with 10% extra, the ground tetracaine available for transfer results in a 6 wt. % composition.

Plasticized base (Jelene) is placed in a 200 ml beaker and the beaker placed directly onto a hotplate. The temperature of the hot plate is set on its lowest position. The plasticized base is gently heated until it became soft and semi-fluid. At which point, the plasticized base is workable for compounding purposes. Plasticized base melts at about 82° F. Heating is stopped before the plasticized base totally melts as separation may occur and plasticized base may not resume its original consistency when cooled.

The ground sodium carboxymethylcellulose is added in small portions to the heated plasticized base with stirring after each addition to ensure a uniform mix. The beaker is removed from the heat and the mixture allowed to cool.

On an ointment slab, a portion of the finely ground tetracaine is worked with PEG400. The tetracaine and PEG400 mixture is combined with a portion of plasticized base via geometric dilution. The cooled plasticized base and sodium carboxymethylcellulose mixture is then worked into the tetracaine, PEG400 and plasticized base mixture via geometric dilution.

In a glass mortar, the ground PolyOx WSR 301 is wetted with PEG400.

The Polyox WSR 301 and PEG400 mixture is then incorporated via geometric dilution with the other ingredients previously mixed together on the ointment slab to form the topical paste.

### Example 2. Treatment of HSV-2 in Human Subjects Using Tetracaine

An otherwise healthy 23 year old female presented pain with urination and a solitary sore on the vulva. It was initially unclear by visible inspection if the solitary sore was herpes or a shave injury. Follow up three days later showed 14-18 painful blisters across her perineum consistent with a primary HSV2 outbreak appearance and distribution. The patient reported Pain 7/10, and worse with urination. Application of a representative pharmaceutical composition of this disclosure as described in Example 1 immediately reduced the patient's reported pain to 1/10. The patient was given the formulation to use at home (two applications per day). The patient was also offered a prescription for 1 gram of Valtrex to be taken twice a day. The patient exclusively self-applied the tetracaine formulation and did not use the Valtrex prescription.

Three days later the patient showed blisters in the final healing stages, with a reported pain 2/10 at max, and no erythema. The patient's surface wounds showed almost complete re-epithelialization.

As a comparison, before the date of the patient's treatment, typical otherwise healthy female patients of this same age presenting similar herpes sores are given a prescription for Valtrex and take up to two weeks to resolve their herpes ulcers. The tetracaine formulation of this disclosure therefore resolved the HSV-2 genital herpes outbreak in a surprisingly less amount of time than the standard of care, Valtrex, or other anti-viral therapies.

A second female human subject of 42 years in age with a long history of HSV-2 who has previously taken oral Valtrex™ (Valacyclovir) to resolve past HSV-2 ulcerations was administered a representative pharmaceutical composition of this disclosure as described in Example 1. The subject has previously resolved HSV-2 ulcerations in 5-7 days after outbreak when administered Valtrex. The subject was administered the representative tetracaine composition twice a day for two days at which point the subject's lesions were completely re-epithelialized and the pain was reduced to 0/10. Before treatment, the subject reported pain at 5/10 at max. The subject identified a long-felt need for the tetracaine-based treatment in stating “Where has this stuff been the last twenty years?”

### Example 3. HSV-2 Cytopathic Effect (CPE) Inhibition Assay Development in RyR1 Expressing Cells

The mechanism of action for Ryanodine receptor antagonism towards inhibiting Herpes Simplex Virus-2 (HSV-2) was studied in HEK 293 cells that express type 1 Ryanodine receptors (HEK293-RyR1). Additionally, a calcium release assay using the Fluo-4 Direct™ Calcium Assay Kit (Invitrogen, F10471) was conducted in parallel to measure HSV-2 mediated calcium flux. The final, developed assay with two concurrent readouts of CPE and Calcium flux was used to assess the mechanism of action of tetracaine as an antagonist of RyR1.

HSV-2 infection was established in HEK293-RyR1 cells by testing three different multiplicities of infection (MOI) as part of the development of the CPE inhibition assay. An inhibitor of HSV-2 was incubated with cells and subsequently infected with HSV-2 at three different MOIs. Cells-only and virus-only controls were included. Cells were observed daily to monitor CPE progression, and the incubation was allowed to continue until sufficient CPE of 60-80% is observed in the virus-only controls. CPE was quantified via crystal violet staining and results given as % inhibition and IC50 values, 50% of the maximum inhibitory dosage of the test article.

In a separate experiment, the Fluo-4 Direct™ Calcium Assay Kit was used to assess calcium response in HEK293-RyR1 cells. HEK293-RyR1 cells were treated with either a calcium agonist, or a calcium antagonist, and the kit used to measure calcium flux as per manufacturer guidelines.

### Materials and Methods

The virus was Herpes Simplex Virus 2, strain MS, lot 7172020, at MOI of 0.01 (ATCC, Catalog #VR-540). HEK293-RyR1 cell line was obtained from Kerafast (Cat. #EUR301). Growth medium was DMEM (High Glucose) supplemented with 10% FBS, 2 mM L-Glutamine, 1% Pen/Strep, 100 g/ml Hygromycin. Incubation medium was DMEM (High Glucose) supplemented with 2% FBS, 2 mM L-Glutamine, 1% Pen/Strep. Infection medium was DMEM (High Glucose) supplemented with 2 mM L-Glutamine, 1% Pen/Strep. Acyclovir (as a control HSV-2 MS inhibitor) was stored at cell culture grade water (Ultrapure DNAse/RNase free distilled water, Thermo Scientific Cat. 10977015) and tested at concentrations of 10, 3.2, 1.0, 0.32, 0.10, 0.032, 0.010, 0.0032 50, 25, 12.5, 6.25, 3.125, 1.5625, 0.78125, 0.3906 100, 50, 25, 12.5, 6.25, 3.125, 1.5625, 0.78125 mg/ml. Carbachol (a Calcium agonist), obtained from Fisher Scientific (ASL0667403) was stored in cell culture-grade water and tested at concentrations of 333, 166.65, 83.325, 41.6625 mg/ml. Tetracaine base USP was formulated in propylene glycol and tested at concentrations of 100, 60, 40, 20, 10, 5, 2, 0.05 mg/mL. Poly-D-lysine was obtained from ThermoFisher (A3890401). Corning 96-well polystyrene tissue culture treated multiwall plates were used. Dulbecco's PBS Saline was obtained from Corning (21-0310CM), heat inactivated fetal bovine serum (Hi-FBS) from Gibco (16410), Fluo-4 Direct™ Calcium Assay Kit (Invitrogen, F10471), 100× Penicillin/Streptomycin (P/S) from Gibco (10378-016), 200 mM L-glutamine from ThermoFisher (25030-164), DMEM Dulbecco's Modified Eagle's Medium (ATCC, 30-2002). A Nexcelcom Mino Cellometer was used for cell counting and measurement. GraphPad Prism 9 was used for data analysis.

### Cytopathic Effect (CPE) Inhibition Assay

### Cell Seeding

HEK293-RyR1 cells in growth medium were seeded in clear poly-d lysine coated 96-well plates at 5×104 cells per well and were incubated overnight (16-20 hours) at 37° C. with 5% CO2.

### Tetracaine Dilution

The next day, cells should appear 70-80% confluent. Pre-warm infection medium to 37° C. Eight two-fold serial dilutions of the inhibitor Acyclovir and tetracaine (TA1) were prepared in dilution plates in incubation medium at four times the final concentration.

### Virus Infection

HSV-2 MS was prepared at the specific MOI in infection medium and added to the acyclovir/TA-containing wells in a dilution block. Then, a total of 200 μL per well of TA/virus mixture was immediately transferred to each well per replicate. Growth medium was aspirated from the cells prior to virus and TA addition. Virus-only control wells were included. Plates were incubated at 37° C. with 5% CO2 until sufficient CPE was observed in the virus-only control wells (˜72 hours). Plates were monitored daily to observe CPE levels.

### Crystal Violet Staining

When visual inspection of the plates showed sufficient CPE (˜72 hours) had been achieved in the virus-only control wells, the plates were terminated. Cells were fixed and stained by adding crystal violet stain directly on top of the inoculum and plates were incubated at room temperature for one hour.

After one hour, plates were washed with tap water and blotted on absorbent paper. Plates were allowed to air dry, and the absorbance of each well was read at 570 nm with a plate reader.

### Data Evaluation

Absorbance values were exported into Excel. Data was normalized to the average absorbance of the virus-only wells, considered to be 0% inhibition, to calculate % inhibition values. The % inhibition values were graphed using a sigmoidal dose response model and IC50 values were derived from the inhibition curves.

### Calcium Release Assay

The Fluo-4 Direct™ Calcium Assay Kit was be used to assess calcium response in RyR1-expressing cells treated with a calcium agonist (Carbachol) or infected with HSV-2 MS at an MOI of 0.01. For the virus-infected cells, procedures as described above were followed but with a 96-well black plate.

### Cell Seeding

Cells were seeded in 100 μL of incubation medium in black 96-well plates coated with poly-D-lysine at 5.0E+04 cells per well and incubated at 37° C. with 5% CO2 overnight (16-20 hours).

### Fluo-4 Direct™ Reagent Preparation

The next day, a 250 mM stock solution of probenecid was prepared by adding 1 mL of Fluo-4 Direct™ calcium assay buffer (labeled Component C) to each 77 mg vial of water-soluble probenecid. The vial was vortexed until dissolved.

A 2× Fluo-4 Direct™ calcium reagent loading solution with 5 mM probenecid was prepared by adding 10 mL of Fluo-4 Direct™ calcium assay buffer and 200 μL of the 250 mM stock solution of probenecid to one bottle of 2× Fluo-4 Direct™ calcium reagent (labeled Component A). The solution was vortexed and allowed to sit for five minutes for complete dissolution.

Solutions of carbachol, a calcium agonist, were prepared in Fluo-4 Direct™ calcium assay buffer without probenecid.

### Fluo-4 Direct™ Calcium Assay

Plated cells were removed from the incubator and 100 μL of incubation medium were added to the plate. Then, 100 μL of 2× Fluo-4 Direct™ calcium reagent loading solution was added. Plates were incubated for 30 minutes at 37° C. with 5% CO2 and for 30 minutes at room temperature. Add 100 μl of the TA dilutions (TA dilutions were done in Fluo-4 Direct calcium assay buffer without probenecid).

The fluorescence of each well was measured directly after the addition of carbachol after setting the fluorescence reader for excitation at 494/10 nm and emission at 520/10 nm.

### Fluorescence Detection

The fluorescence of each well was measured directly after the addition of carbachol with the Biotek Cytation 5 plate reader using the fluorescence reader settings for excitation at 494/10 nm and emission at 520/10 nm. The fluorescence measurements in relative fluorescence units (RFU) were recorded and exported to Excel.

### Data Evaluation

ΔRFU is given in relative fluorescent units (RFU) as the maximum response (TA or virus treated cells) minus the minimum response (cells only).

Cytopathic Effect (CPE) Inhibition Assay with Calcium release assay using representative Tetracaine Formulation

### Cell Seeding

HEK293-RyR1 cells in growth medium were seeded in clear (CPE assay) or black (Calcium release assay) poly-d lysine coated 96-well plates at 5.0×104 cells per well and were incubated overnight (16-20 hours) at 37° C. with 5% CO2.

### Test Article and Virus Dilution

The next day, eight two-fold serial dilutions of the inhibitor Acyclovir were prepared in dilution plates in incubation medium at four times the final concentration. Dilutions of the client TA tetracaine were prepared in dilution plates in incubation medium at four times the final concentration. HSV-2 MS was added at MOI of 0.01 to the TA dilution plate. Virus and TA dilutions are immediately added to the plate.

Virus and TA addition to the cells.

Growth medium was aspirated from the cells and 200 μL TA/virus dilutions were added in triplicate. Cells-only and virus-only control wells were included and received 200 μL incubation/infection medium only. Cells only that receive treatment of carbachol were also added to the plate plan to ensure that the Calcium release assay worked. These cells received 200 μL incubation/infection medium only.

Plates were incubated at 37° C. with 5% CO2 until sufficient CPE was observed in the virus-only control wells. Plates were monitored daily to observe CPE levels.

### Crystal Violet Staining

When visual inspection of the plates showed sufficient CPE had been achieved in the virus-only control wells, the plates were terminated. Cells were fixed and stained by adding crystal violet stain directly on top of the inoculum and plates were incubated at room temperature for one hour.

After one hour, plates were washed with tap water and blotted on absorbent paper. Plates were allowed to air dry, and the absorbance of each well was read at 570 nm with a plate reader.

### Data Evaluation

Absorbance values were exported into Excel. Data was normalized to the average absorbance of the virus-only wells, considered to be 0% inhibition, to calculate % inhibition values. The % inhibition values were graphed using a sigmoidal dose response model and IC50 values were derived from the inhibition curves.

### Reagent Preparation for Fluo-4 Direct™ Calcium Assay

On day 3, a 250 mM stock solution of probenecid was prepared by adding 1 mL of Fluo-4 Direct™ calcium assay buffer (labeled Component C) to each 77 mg vial of water-soluble probenecid. The vial was vortexed until dissolved.

A 2× Fluo-4 Direct™ calcium reagent loading solution with 5 mM probenecid was prepared by adding 10 mL of Fluo-4 Direct™ calcium assay buffer and 200 μL of the 250 mM stock solution of probenecid to one bottle of 2× Fluo-4 Direct™ calcium reagent (labeled Component A). The solution was vortexed and allowed to sit for five minutes for complete dissolution.

Solutions of carbachol, a calcium agonist, were prepared in Fluo-4 Direct™ calcium assay buffer without probenecid.

### Fluo-4 Direct™ Calcium Assay

Plated cells were removed from the incubator. TA/virus dilutions were removed and 100 μL of incubation medium was added to the plate. Then, 100 μL of 2× Fluo-4 Direct™ calcium reagent loading solution was added. Plates were incubated for 30 minutes at 37° C. with 5% CO2 followed by 30 minutes at room temperature. Carbachol dilutions (100 μL) were added to the plate that contains only cells as a positive control.

The fluorescence of each well was measured directly after the addition of carbachol after setting the fluorescence reader for excitation at 494/10 nm and emission at 520/10 nm.

### Fluorescence Detection

The fluorescence of each well was measured directly after the addition of carbachol with the Biotek Cytation 5 plate reader using the fluorescence reader settings for excitation at 494/10 nm and emission at 520/10 nm. The fluorescence measurements in relative fluorescence units (RFU) were recorded and exported to Excel.

### Data Evaluation

ΔRFU is given in relative fluorescent units (RFU) as the maximum response (TA or virus treated cells) minus the minimum response (cells only).

### Results

### CPE Inhibition Assay Optimization

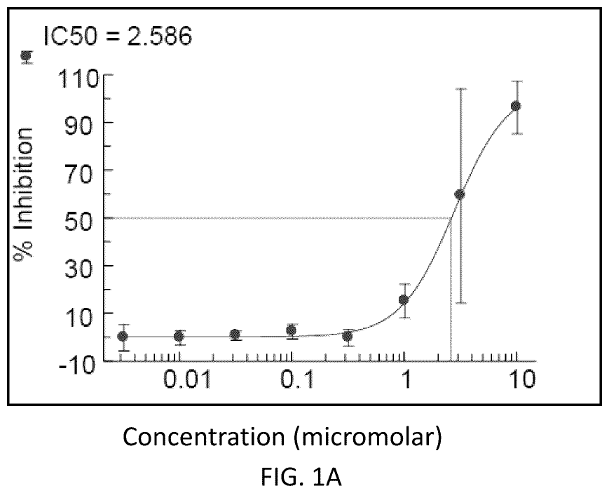

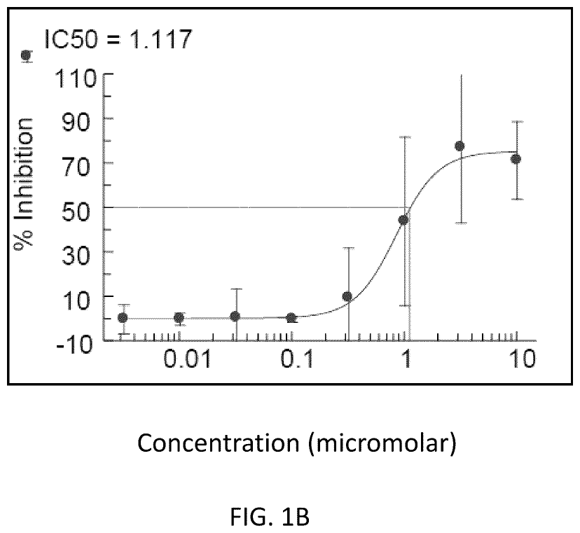

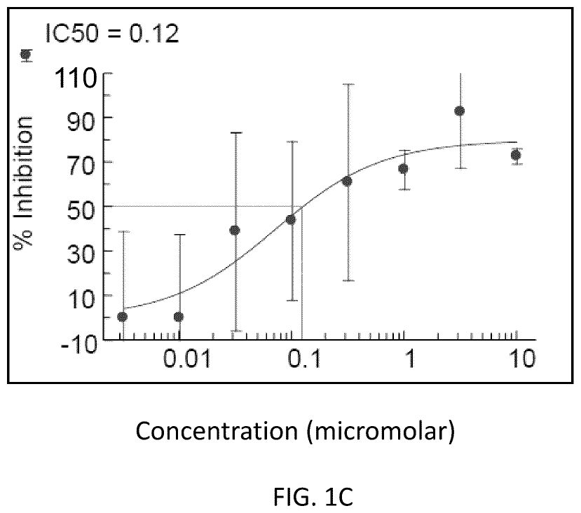

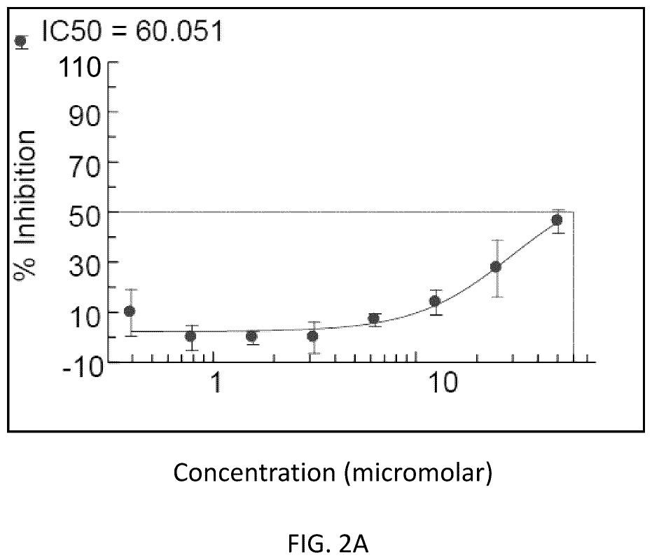

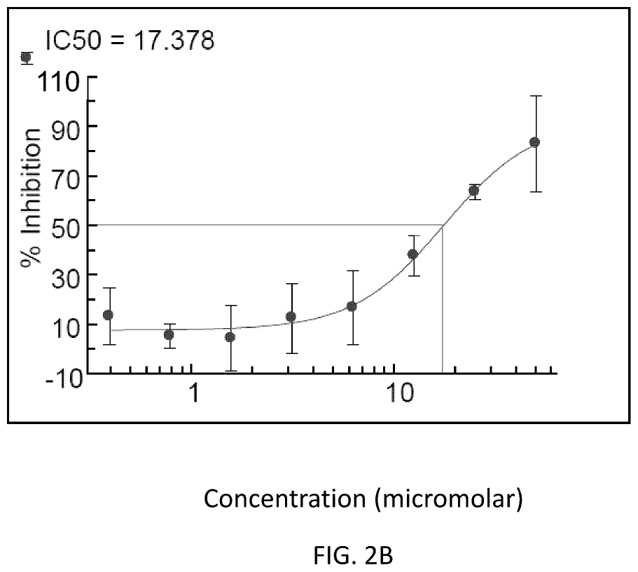

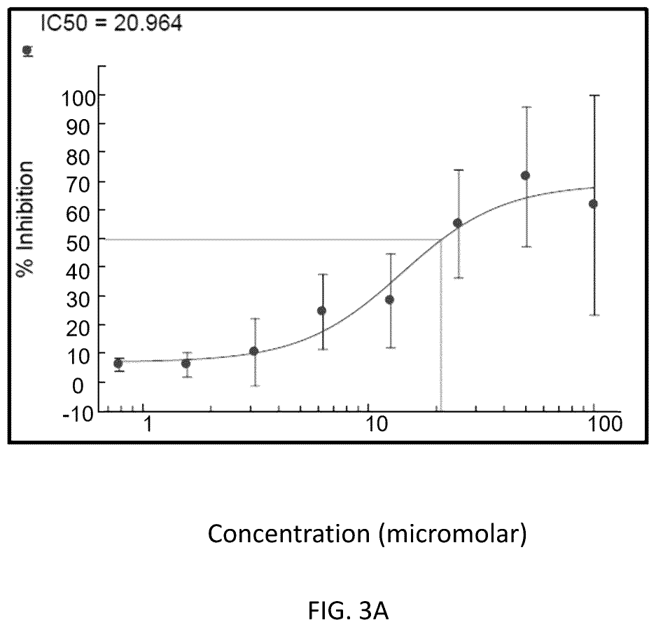

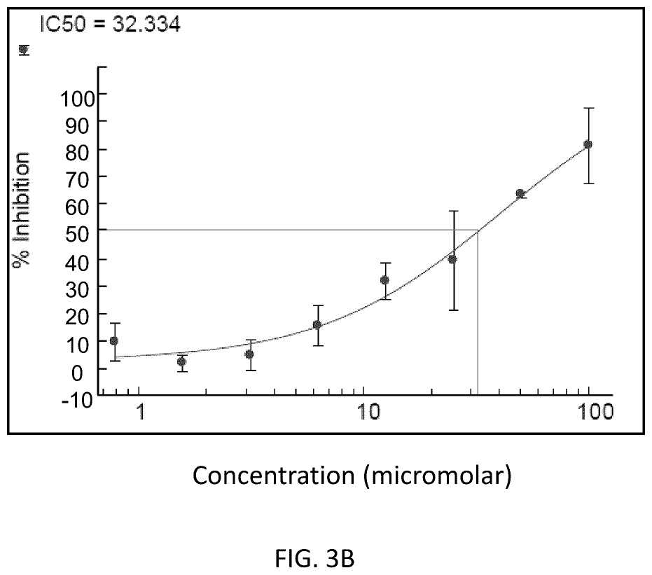

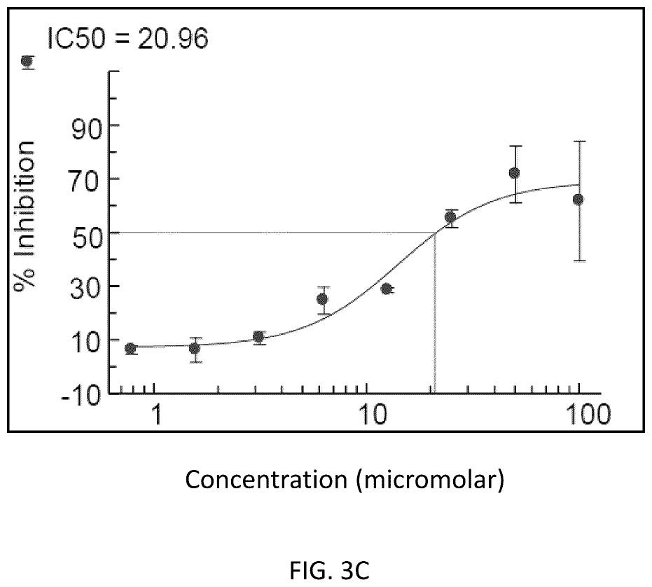

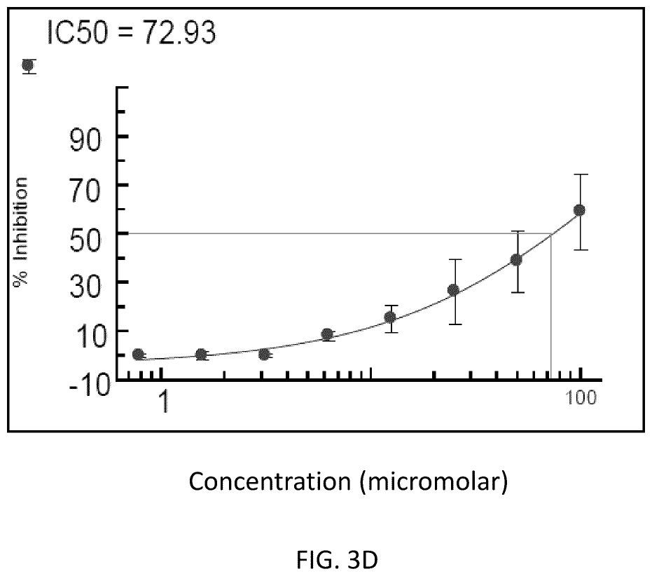

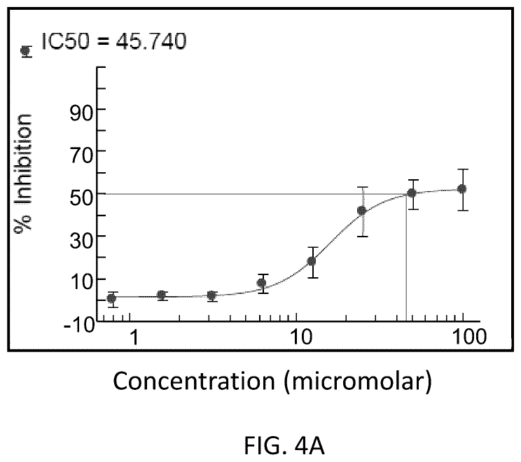

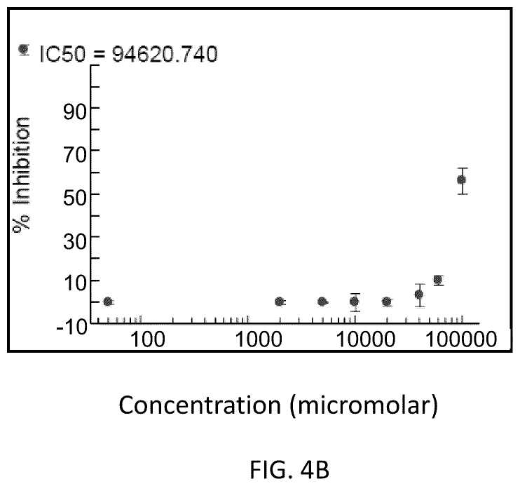

HSV-2 infection was established in cells expressing RyR1 receptors. The multiplicity of infection (MOI) was optimized by testing three MOI conditions 0.01, 0.005, and 0.001 (FIG. 1). The MOI that exhibited the greatest infection was 0.01. The cell seeding density and starting concentration of acyclovir of the assay was also optimized (FIG. 2 and FIG. 3) and further studies were conducted using a starting concentration of 100 μg/mL of acyclovir and a seeding density of 5.0×104 cells per well.

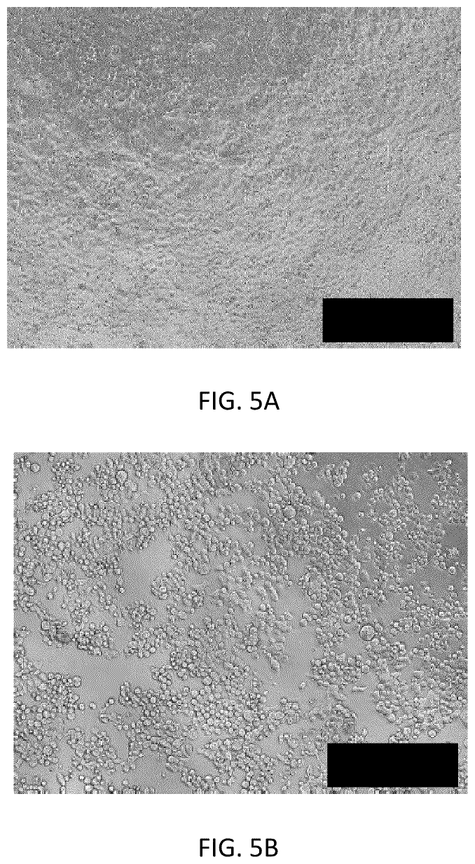

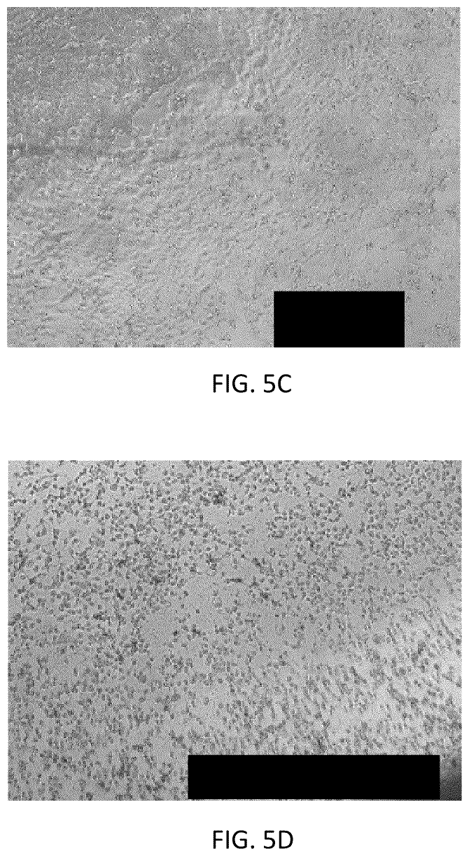

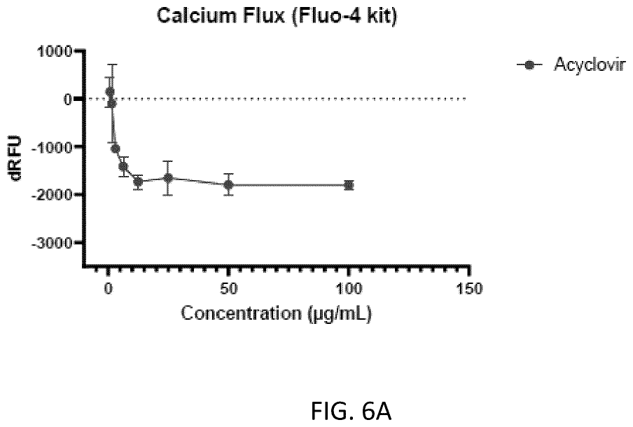

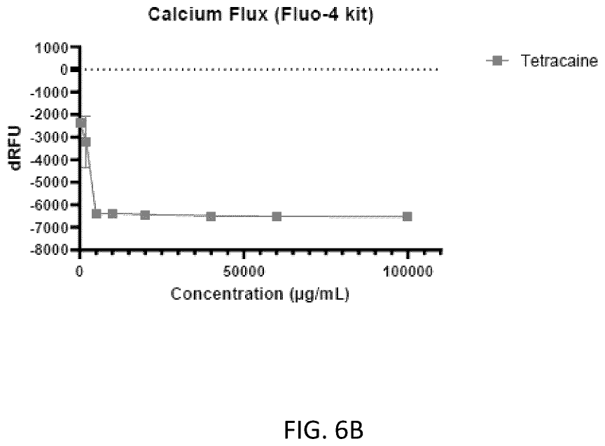

HSV-2 infection was established in cells expressing RyR1 receptors at an MOI of 0.01. A starting concentration of 100 μg/mL of acyclovir (positive control) and a seeding density of 5.0×104 cells per well was used. Tetracaine was tested at 100, 60, 40, 20, 10, 5, 2 and 0.05 mg/mL. The IC50s of Acyclovir and Tetracaine were 45.7 g/mL and 94,620 g/mL, respectively. Abnormal cell morphology was also noted in cells treated with Tetracaine (FIG. 5D) compared to cells only (FIG. 5A), virus-only cells (FIG. 5B) and Acyclovir-treated (FIG. 5C) cells. A parallel experiment was conducted with similar concentrations of Acyclovir and Tetracaine to detect calcium flux. The relative fluorescence units (RFU) were used to calculate the difference (designated as ΔRFU) between the maximum response (TA or virus treated cells) minus the minimum response (cells only). In this assay, negative ΔRFU values indicate intracellular calcium mobilization. Cells treated with Acyclovir showed a maximum average ΔRFU of −1801.6 at the highest concentration of acyclovir (FIG. 6A). Cells treated with tetracaine showed a 3.6-fold higher average ΔRFU of −6527.9 at the highest concentration of tetracaine (TA) tested suggesting a much higher intracellular calcium concentration (FIG. 6B).

Assay development activities for set up of calcium flux assay and HSV-2 CPE inhibition assay in RyRy1 cells was completed with assay parameters identified for cell seeding density, HSV-2 viral infectivity and incubation periods for optimal readout. These activities were performed without incidence. Tetracaine along with Acyclovir, an approved drug and known inhibitor of HSV-2 was tested in both assays showing inhibition of infection at a defined inhibition constant and higher intracellular calcium concentrations in infected cells treated with acyclovir and Tetracaine. The results of these experiments demonstrate that tetracaine is an effective antagonist of Ryanodine receptors, and also a useful agent for treating HSV-2 infection, including by killing HSV-2 viral particles and/or reducing HSV-2 viral load.

### Example 4. Demonstration of Representative Compositions for the Treatment of Genital Herpes in Animal Models

As further evidence of the surprising effectiveness and rapid treatment of the representative Ryanodine receptor antagonist (e.g., tetracaine) compositions of this disclosure for the treatment of genital herpes, animal models can be performed according to the procedures described in Hook et al., Curr Protoc. 2021 Dec; 1(12): e332. doi: 10.1002/cpz1.332, herein incorporated by reference. Guinea pigs and mice models can be created for genital herpes. Application of the composition described in Example 1 is performed on a first cohort of models. Application of the standard of care (e.g., Valtrex™) on another cohort is performed on a second cohort of models. Application of the carrier (the composition of Example 1, but lacking in tetracaine) is performed on a third cohort of models. The experiments are expected to demonstrate that the cohort treated with the composition of Example 1 will resolve the genital herpes outbreak (e.g., ulcers) much more quickly than those of the Valtrex™ or vehicle control cohorts, thereby demonstrating the effectiveness of the tetracaine compositions of this disclosure for treating subjects with HSV-2.

The inventions described and claimed herein have many attributes and embodiments including, but not limited to, those set forth or described or referenced in this Detailed Disclosure. It is not intended to be all-inclusive and the inventions described and claimed herein are not limited to or by the features or embodiments identified in this Detailed Disclosure, which is included for purposes of illustration only and not restriction. A person having ordinary skill in the art will readily recognise that many of the components and parameters may be varied or modified to a certain extent or substituted for known equivalents without departing from the scope of the invention. It should be appreciated that such modifications and equivalents are herein incorporated as if individually set forth. The invention also includes all of the steps, features, compositions and compounds referred to or indicated in this specification, individually or collectively, and any and all combinations of any two or more of said steps or features.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents. Reference to any applications, patents and publications in this specification is not, and should not be taken as, an acknowledgment or any form of suggestion that they constitute valid prior art or form part of the common general knowledge in any country in the world.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants. Furthermore, titles, headings, or the like are provided to enhance the reader's comprehension of this document, and should not be read as limiting the scope of the present invention. Any examples of aspects, embodiments or components of the invention referred to herein are to be considered non-limiting.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A method of treating HSV-2 associated genital herpes in a subject, the method comprising topically administering a therapeutically effective amount of a composition comprising one or a plurality of Ryanodine receptor antagonists to the subject,
wherein the composition does not include an aqueous component, and
wherein the Ryanodine receptor antagonist is the sole active ingredient in the composition.

2. A method of reducing the number of ulcers associated with genital herpes in a subject, the method comprising topically administering a therapeutically effective amount of a composition comprising one or a plurality of Ryanodine receptor antagonists to the subject,
wherein the composition does not include an aqueous component, and
wherein the Ryanodine receptor antagonist is the sole active ingredient in the composition.

3. A method of inhibiting the replication of a HSV-2 in a subject, the method comprising administering a therapeutically effective amount of a composition comprising one or a plurality of Ryanodine receptor antagonists to the subject,
wherein the composition does not include an aqueous component, and
wherein the Ryanodine receptor antagonist is the sole active ingredient in the composition.

4. The method of claim 1, wherein the Ryanodine receptor antagonist is selected from: Tetracaine, procaine, Dantrolene, Chlorantraniliprole, cyantraniliprole, flubendiamide, cyclaniliprole, tetraniliprole, Ryanodine, JTV 519 fumarate ((4-[3(1-(4-benzyl)piperidinyl)propionyl]-7-methoxy-2,2,4,5-tetrahydro-1,4-benzothiazepine, fumarate salt), Ruthenium Red, DHBP (1,1′-diheptyl-4,4′-bipyridium), VK-II-86, Phenytoin (diphenylhydantoin), Flecainide, Carvedilol, EL20 (2-(diethylamino)ethyl 4-(butylamino)-2-methoxybenzoate), Xanthotoxol, 5-hydroxy-1,4-naphthalenedione, Rycal (ARM210) (Benzoic acid, 4-((2,3-dihydro-7-methoxy-1,4-benzothiazepin-4(5H)-yl)methyl)-), 4-(2-aminopropyl)-3,5-dichloro-N,N-dimethylaniline (FLA 365), 2-(dimethylamino)ethyl 4-(butylamino)-5-chloro-2-methoxybenzoate (EL1), 2-(dibutylamino)ethyl 4-(butylamino)-2-methoxybenzoate (EL2), 2-(dibutylamino)ethyl 4-(butylamino)-5-chloro-2-methoxybenzoate (EL3), 2-(diethylamino)ethyl 4-(butylamino)-3-methoxybenzoate (EL4), 2-(dimethylamino)ethyl 4-(butylamino)-2-methoxybenzoate (EL5), 4-(butylamino)-3-methoxybenzoic acid (EL6), 2-(dimethylamino)ethyl 4-(butylamino)-3-methylbenzoate (EL7), 2-(diethylamino)ethyl 4-(butylamino)-3-methylbenzoate (EL8), and 2-(diethylamino)ethyl 4-(butylamino)-5-chloro-2-methoxybenzoate (EL9).

5. The method of claim 4, wherein the Ryanodine receptor antagonist is tetracaine.

6. The method of claim 5, wherein the tetracaine is substantially in the deprotonated base form as tetracaine base.

7. The method of claim 1, wherein the composition further comprises PEG400 (polyethylene glycol, mean average molecular weight of 400).

8. (canceled)

9. The method of claim 1, wherein the number of ulcers is reduced to zero within five days after the initial administration of the composition.

10. The method of claim 1, wherein the composition comprises from about 2 to about 8 wt. % tetracaine.

11. The method of claim 10, wherein the composition comprises from about 4.0 to about 6.0 wt. % tetracaine.

12. The method of claim 11, wherein the composition comprises about 6 wt. % tetracaine.

13. The method of claim 1, wherein the composition comprises a carrier comprising a non-aqueous vehicle.

14. The method of claim 1, wherein the composition is topically applied up to three times a day.

15. The method of claim 14, wherein the composition is topically applied twice daily.

16. The method of claim 14, wherein the subject presents a herpes ulcer before treatment and the composition is topically applied to said ulcer.

17. (canceled)

18.-20. (canceled)
